(19) United States
(12) Patent Application Publication
(10) Pub. No.: US 2015/0202179 A1
(43) Pub. Date: Jul. 23, 2015

(54) FATTY ACID DERIVATIVES FOR USE IN A METHOD OF TREATING DEPRESSION AND ASSOCIATED CONDITIONS

(71) Applicants: YISSUM RESEARCH DEVELOPMENT COMPANY OF THE HEBREW UNIVERSITY OF JERUSALEM LTD., JERUSALEM (IL); ALQUDS UNIVERSITY, JERUSALEM (IL)

(72) Inventors: Elliot Berry, Jerusalem (IL); Yosefa Avraham, Jerusalem (IL); Joshua Katzhendler, Jerusalem (IL); Josef Mograbi, Tel Aviv (IL); Yousef Najajreh, Beit Jalla (IL); Lia Vorobeiv, Jerusalem (IL)

(21) Appl. No.: 14/415,863

(22) PCT Filed: Jul. 21, 2013
(86) PCT No.: PCT/IL2013/050618

§ 371 Date: Jan. 20, 2015

Related U.S. Application Data
(60) Provisional application No. 61/673,925, filed on Jul. 20, 2012.
(60) Provisional application No. 61/674,525, filed on Jul. 23, 2012.

(51) Int. Cl. A61K 31/325; A61K 31/17; A61K 31/165; A61K 31/16

(52) U.S. Cl. CPC A61K 31/325; A61K 31/16; A61K 31/17; A61K 31/165

(57) ABSTRACT

The invention provides fatty acid derivatives for use in a method of treatment of at least one disease, disorder or condition selected from anxiety, depression, conditions associated menopause, stress, bipolar disorder, neuropathic pain and fibromyalgia.

### TECHNOLOGICAL FIELD

The present invention is directed to the filed of treatment of depression and associated conditions using compounds that are fatty acid derivatives.

### BACKGROUND

Depression is a chronic, recurring and potentially life-threatening illness that affects up to 20% of the population across the world. The incidence of depression (and also other mental disorders) is high in the United States and worldwide, and the inadequacy of currently available treatments contributes to the significant health burden associated with depression. Despite its prevalence and considerable impact on human, only little is known about its pathogenesis. One of the major reasons is the restricted availability of relevant validated animal models. The lack of animal models is mainly because some core symptoms such as depressed mood, feeling of worthlessness, and recurring thoughts about death or committing suicide, are impossible to be modeled on laboratory animals.

WO2009109973 discloses use of new and known amino acid derivatives of fatty acids useful for treating or reducing the severity of obesity, increasing or facilitating weight loss, suppressing appetite, decreasing food consumption and improving cognitive function.

Abraham Y. et al. (J. Med. Chem. 56, 1811-1829 (2013)) disclosed acylethanolamide derivatives that modulate body weight through enhancement of hypothalamic pro-opiomelanocortin (POMC) and/or decreased neuropeptide Y (NPY). Receptor binding indicated that no compound activated CB1, CB2, PPARα, or TRPV1 receptors. Hypothalamic RT-PCR showed that mRNA expression of the anorexigenic genes POMC and CART was up-regulated, while that of the orexigenic genes NPY and CaMKK2 was down-regulated.

### GENERAL DESCRIPTION

The present application provides a compound having the general formula (I):

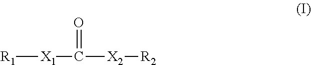

wherein

R₁ is selected from the group consisting of C₁₀-C₂₀ straight or branched alkyl, C₁₀-C₂₀ straight or branched alkenyl and C₁₀-C₂₀ straight or branched akynyl;

R₂ is C₂-C₈ straight or branched alkyl-OH; optionally substituted by at least one substituent selected from phenyl, phenyl-OH, —NH₂, —C(═O)O⁻, —NH₃⁺, —C(═O)NH₂, —SH, —SeH, —SCH₃, indolyl, toluyl, imidazoyl, pyrrolidinyl, and guanidinyl;

X₁ and X₂ are each selected from the group consisting of a bond, —O—, —NH—;

provided that at least one of X₁ and X₂ is —NH—;

for use in a method of treatment of at least one disease, disorder or condition selected from anxiety, depression (including post partum depression) conditions associated menopause, stress, bipolar disorder, neuropathic pain and fibromyalgia.

In some embodiments, said compounds defined herein above by general Formula (I) are for use in a method of treatment of at least one disease, disorder or condition selected from anxiety, depression and stress.

In a further aspect of the invention there is provided a compound of general formula (I):

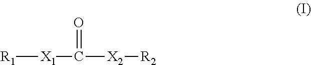

wherein

R₁ is selected from the group consisting of C₁₀-C₂₀ straight or branched alkyl, C₁₀-C₂₀ straight or branched alkenyl and C₁₀-C₂₀ straight or branched akynyl;

R₂ is C₂-C₈ straight or branched alkyl-OH; optionally substituted by at least one substituent selected from phenyl, phenyl-OH, —NH₂, —C(═O)O⁻, —NH₃⁺, —C(═O)NH₂, —SH, —SeH, —SCH₃, indolyl, toluyl, imidazoyl, pyrrolidinyl and guanidinyl;

X₁ and X₂ are each selected from the group consisting of a bond, —O—, —NH—;

provided that at least one of X₁ and X₂ is —NH—;

for use in a method of treatment of a disorder, disease or condition that benefits from raise or enhancement in at least one of leptin and POMC levels of said subject.

The term “C₁₀-C₂₀ straight or branched alkyl” is meant to encompass any saturated hydrocarbon chain having between 10 to 20 carbon atoms that may be either straight or branched (by one or more hydrocarbon branches) at any point on the chain.

The term “C₁₀-C₂₀ straight or branched alkenyl” is meant to encompass any unsaturated hydrocarbon chain having between 10 to 20 carbon atoms that may be either straight or branched (by one or more hydrocarbon branches) at any point on the chain, having at least one double bond between any two carbon atoms on the chain (or branch).

The term “C₁₀-C₂₀ straight or branched alkynyl” is meant to encompass any unsaturated hydrocarbon chain having between 10 to 20 carbon atoms that may be either straight or branched (by one or more hydrocarbon branches) at any point on the chain, having at least one triple bond between any two carbon atoms on the chain (or branch).

The term “C₂-C₈ straight or branched alkyl-OH” (also C₂-C₈ straight or branched alkynol) is meant to encompass any saturated hydrocarbon chain having between 2 to 8, (in some embodiments between 3 to 8) carbon atoms wherein at least one hydrogen is substituted with an —OH group at any position on the alkyl chain (or branch).

The term “optionally substituted” is meant to encompass that the alkyl-OH defined herein above may be substituted (i.e. at least one of the hydrogen atoms of the alkyl-OH is substituted) at any position on the chain or branch by at least one substituent phenyl, phenyl-OH, —NH₂, —C(═O)O⁻, —NH₃⁺, —C(═O)NH₂, —SH, —SeH, —SCH₃, indolyl, toluyl, imidazoyl, pyrrolidinyl and guanidinyl.

It is to be noted that substituents X₁ and X₂ are each selected from the group consisting of a bond, —O—, —NH—; provided that at least one of X₁ and X₂ is —NH—. In some embodiments X₁ or X₂ are a bond, thus connecting R₁ or R₂ (respectively) to the C═O group.

It should be noted that the use of the terms “raise” and/or “enhancement of” levels of the mentioned elements refers to any raise or enhancement in their levels (qualitative and/or quantitative) that may have an effect on a condition of a subject treated.

In some embodiments R₁ is a straight C₁₀-C₂₀ alkenyl. In other embodiments, said alkenyl has a single unsaturated double bond. In further embodiments, said alkenyl has two unsaturated double bonds.

In some embodiments, X₁ is a bond. In other embodiments, X₂ is a bond.

In some embodiments X₁ is —O— or —NH. In other embodiments, X₂ is —NH.

In some other embodiments R₂ is C₃-C₅ straight or branched alkyl-OH. In some further embodiments, R₂ is C₃-C₅ branched alkyl-OH optionally substituted by at least one substituent selected from phenyl, phenyl-OH, —NH₂, —C(═O)O⁻, —NH₃⁺, —C(═O)NH₂, —SH, —SeH, —SCH₃, indolyl, toluyl, imidazoyl, pyrrolidinyl, and guanidinyl. In some other embodiments, R₂ is C₃-C₅ branched alkyl-OH optionally substituted by at least one substituent selected from phenyl, phenyl-OH.

In some embodiments a compound according to any one of the preceding aspects and embodiments is selected from:
1-Oleoyl-L-Valinol amide (Compound 1 or Compound X)
Linoleoyl-L-Leucinol amide (Compound 4)
Oleoyl-L-Leucinol amide (Compound 5)
Elaidoyl-L-Valinol amide (Compound 7)
Elaidoyl-D-Valinol amide (Compound 7A)
Linoleoyl-L-Valinol amide (Compound 10)
Oleoyl-D-Valinol (Compound 11)
Oleyloxycarbonyl-L-Valinol amide (Compound 12 or Compound Y(L))
Oleyloxycarbonyl-D-Valinol amide (Compound 13 or Compound Y(D))
Oleyl-amine-carbonyl-D-valinol (Compound 14)
Oleyl-amine-carbonyl-L-valinol (Compound 15)
Oleylamino-D-2-hydroxyvaline amide (Compound 19)
Oleoyl-L-phenyl alaninol (Compound 26)

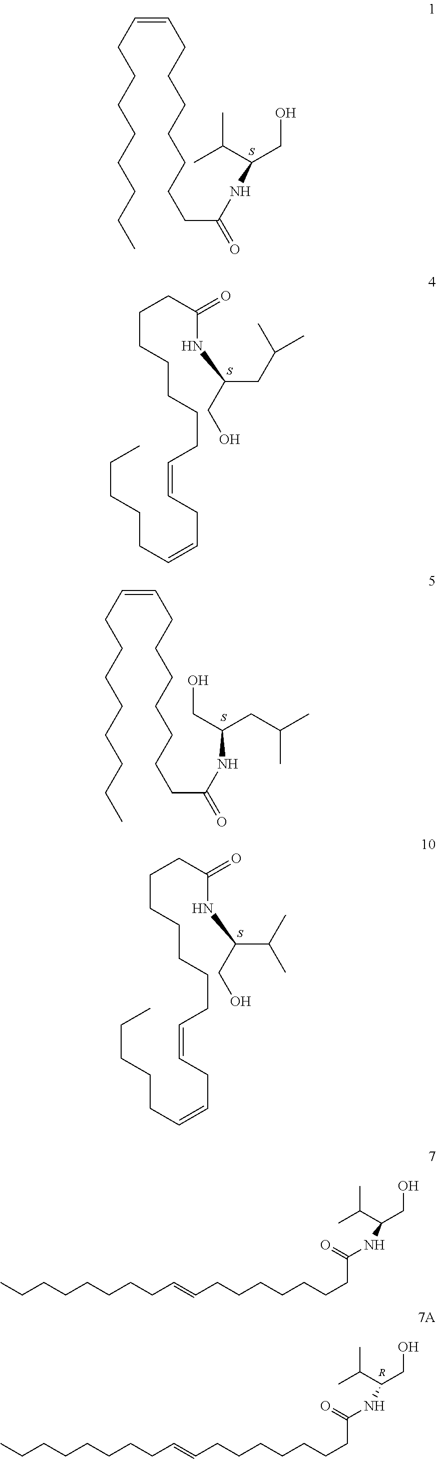

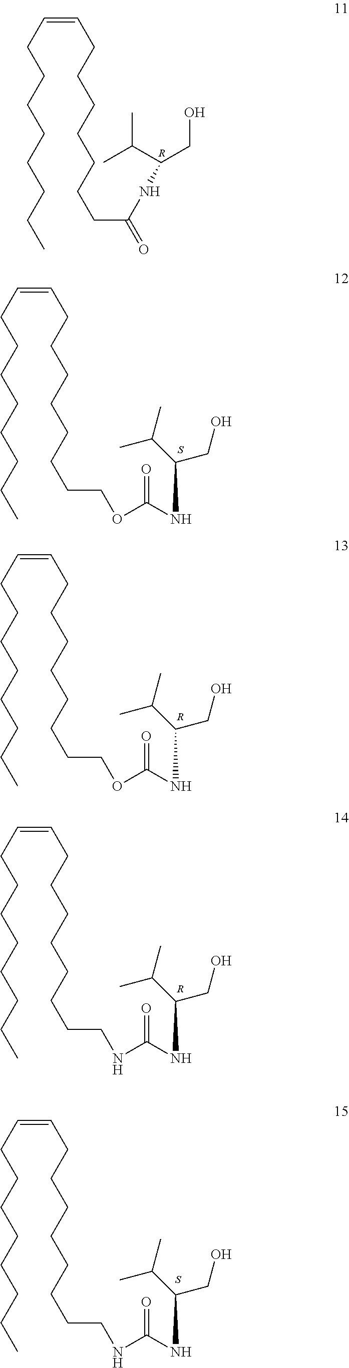

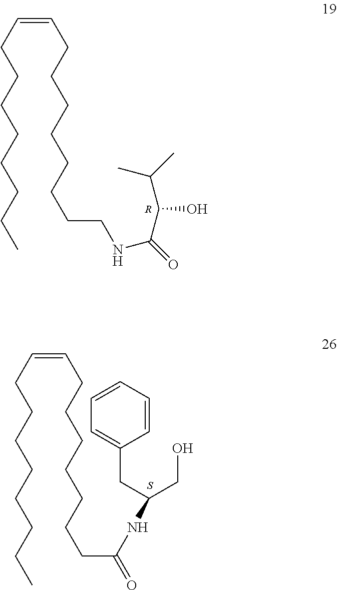

In some further embodiments a compound according to any one of the preceding aspects and embodiments is used in a method for treating depression.

A method of treating a disorder, disease or condition selected from anxiety, depression (including post partum depression) conditions associated menopause, stress, bipolar disorder, neuropathic pain and fibromyalgia, comprising administering to a subject in need thereof a compound having the general formula (I):

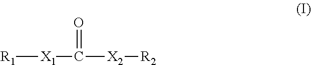

wherein

R₁ is selected from the group consisting of C₁₀-C₂₀ straight or branched alkyl, C₁₀-C₂₀ straight or branched alkenyl and C₁₀-C₂₀ straight or branched akynyl;

R₂ is C₂-C₈ straight or branched alkyl-OH; optionally substituted by at least one substituent selected from phenyl, phenyl-OH, —NH₂, —C(═O)O⁻, —NH₃⁺, —C(═O)NH₂, —SH, —SeH, —SCH₃, indolyl, toluyl, imidazoyl, pyrrolidinyl and guanidinyl;

X₁ and X₂ are each selected from the group consisting of a bond, —O—, —NH—;

provided that at least one of X₁ and X₂ is —NH—.

A method of treating a disorder, disease or condition that benefits from raise in leptin or POMC levels, said method comprising administering to a subject in need thereof a compound having the general formula (I):

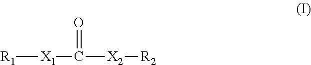

wherein

R₁ is selected from the group consisting of C₁₀-C₂₀ straight or branched alkyl, C₁₀-C₂₀ straight or branched alkenyl and C₁₀-C₂₀ straight or branched akynyl;

R₂ is C₂-C₈ straight or branched alkyl-OH; optionally substituted by at least one substituent selected from phenyl, phenyl-OH, —NH₂, —C(═O)O⁻, —NH₃⁺, —C(═O)NH₂, —SH, —SeH, —SCH₃, indolyl, toluyl, imidazoyl and guanidinyl;

X₁ and X₂ are each selected from the group consisting of a bond, —O—, —NH—;

provided that at least one of X₁ and X₂ is —NH—

A method of raising at least one of leptin and POMC levels in a subject in need thereof, comprising administering to a subject in need thereof a compound having the general formula (I):

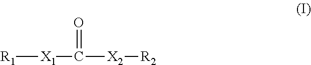

wherein

R₁ is selected from the group consisting of C₁₀-C₂₀ straight or branched alkyl, C₁₀-C₂₀ straight or branched alkenyl and C₁₀-C₂₀ straight or branched akynyl;

R₂ is C₂-C₈ straight or branched alkyl-OH; optionally substituted by at least one substituent selected from phenyl, phenyl-OH, —NH₂, —C(═O)O⁻, —NH₃⁺, —C(═O)NH₂, —SH, —SeH, —SCH₃, indolyl, toluyl, imidazoyl, pyrrolidinyl and guanidinyl;

X₁ and X₂ are each selected from the group consisting of a bond, —O—, —NH—; provided that at least one of X₁ and X₂ is —NH—.

In some embodiments, a compound of the invention is administered in a dose of between about 0.1 mg/Kg to about 10 mg/Kg. In some other embodiments, a compound of the invention is administered in a dose of between about 1 mg/Kg to about 10 mg/Kg.

It should be understood that in case a compound disclosed in the present invention contain at least one chiral stereogenic center, and thus may exist in, and be isolated/synthesized as, enantiomeric (as R or S/D or L enantiomers) or diastereomeric forms, or as racemic or non-racemic mixtures of enantiomers. The present invention includes any possible enantiomers, diastereomers, racemates or mixtures of any compound of the general Formula (I). Where the herein-described processes for the preparation of the compounds of use in the invention give rise to mixtures of stereoisomers, these isomers may be separated by conventional techniques, such as preparative chromatography. The compounds may be prepared in racemic form, or individual enantiomers may be prepared either by enantiospecific synthesis or by chiral chromatographic separation of a racemate.

In a further aspect of the invention there is provided compounds of Formula (I) as disclosed herein above for use in a method of enhancing at least one of norepinephrine (NE) and serotonin (5HT) levels in at least one of the hypothalamus and the hippocampus. The invention therefore provides compounds of Formula (I), as disclosed herein above for use in a method of treatment of a disorder, disease or condition that benefits from raising or enhancing at least one of norepinephrine (NE) and serotonin (5HT) levels in at least one of the hypothalamus and the hippocampus.

In some embodiments the following compounds:

oleoyl-L-valinol amide (compound 1 and compound X);

oleylaminecarbonyl-D-valinol (compound 14) and

oleylaminecarbonyl-L-valinol (compound 15)

are used in a method of enhancing both norepinephrine (NE) and serotonin (5HT) levels in the hypothalamus and hippocampus.

In other embodiments, the following compounds:

linoleoyl-L-Leucinol amide (compound 4),

linoleoyl-L-Valinol amide (compound 10),

oleyloxycarbonyl-L-valinol amide (compound 12)

oleyloxycarbonyl-D-valinol amide (compound 13)

oleylamino-D-2-hydroxy valine amide (compound 19)

are used in a method of enhancing both norepinephrine (NE) and serotonin (5HT) levels in the hippocampus.

In further embodiments, the compound oleoyl-L leucinol amide (compound 5) is used in a method of enhancing 5-HT levels in the hippocampus.

In other embodiments, the following compounds:

oleoyl-D-valinol amide (compound 11),

oleyloxycarbonyl-L-valinol amide (compound 12),

oleyloxycarbonyl-D valinol amide (compound 13),

are used in a method of enhancing serotonin (5HT) levels in the hypothalamus.

In some further embodiments, the compound elaidoyl-L-valinol amide (compound 7), is used in a method of enhancing 5-HT levels in both hippocampus and hypothalamus

In some further embodiments, the compound oleoyl-L-alaninol (compound 26), is used in a method of enhancing NE in the hippocampus.

Thus, it was found that all the above mentioned compounds may be used for the treatment and/or prevention of depression and/or depression associated disorders. Additionally, all the above mentioned compounds may be used for the treatment and/or prevention of fibromyalgia, neuropathic pain relief, and for the relief of menopausal symptoms.

The present invention is also based on the finding that compounds of general Formula (I) as disclosed herein (such as for example compound 1, 12 and 15) enhance leptin levels, injection of the mentioned compounds to Sabra mice caused significant decrease in food consumption and weight however administration of the same compounds to OB/OB mice which lack the leptin gene did not change either food consumption or weight. Thus the above mentioned compounds are mediated by the enhancement of leptin levels which is a survival and anti-depression hormone.

In a further aspect of the invention there is provided compounds of Formula (I) as disclosed herein above for use in a method of treating a condition wherein enhancing at least one of leptin levels and POMC expression is beneficial.

In some further embodiments, the following compounds:

oleyl-L-valinol amide (compound 1 or compound X);

oleyl-oxycarbonyl-L-valinol amide (compound 12) and

oleyl-amine-carbonyl-L-valinol (compound 15)

are used in a method of enhancing brain leptin levels.

In some further embodiments, the following compounds:

oleyl-L-valinol (compound 1 and compound X);

oleyl-L-leucinol amide (compound 5),

stearoyl-L-valinol amide (compound 8)

oleoyl-D-valinol amide (compound 11),

oleyloxycarbonyl-L-Valinol amide (compound Y or compound 12),

oleyloxycarbonyl-D-Valinol amide (compound 13)

oleylaminecarbonyl-D-valinol (compound 14),

oleyl-L-hydroxylvaline (compound 18)

are used in a method of enhancing POMC expression in the hypothalamus.

The term “anxiety” is meant to encompass any type and severity of unpleasant state of inner emotional instability, including anxiety disorder, often accompanied by nervous behavior and any condition or disorder associated therewith (such as for example restlessness, fatigue, problems in concentration, and muscular tension).

The term “depression” is meant to encompass any type and severity of low mood and aversion to activity that can affect a person's thoughts, behavior, feelings and sense of well-being. The term includes any condition or disorder associated therewith, including, but not limited to sadness, feel of emptiness, hopelessness, worried, helpless, worthless, guilt, irritability, hurt, or restlessness, lose of interest in activities, experience loss of appetite or overeating, problems concentrating, remembering details, or making decisions, and may lead to attempted suicide, insomnia, excessive sleeping, fatigue, loss of energy, or aches, pains, or digestive problems.

The term “stress” is meant to encompass any type and severity of strain and pressure feelings, including any disorders and conditions associated therewith including a sense of being overwhelmed, feelings of anxiety, overall irritability, insecurity, nervousness, social withdrawal, loss of appetite, depression, panic attacks, exhaustion, high or low blood pressure, skin eruptions or rashes, insomnia, migraine, gastrointestinal difficulties (constipation or diarrhea), and for women, menstrual symptoms, heart problems.

The term “bipolar disorder” (also known as bipolar affective disorder, manic-depressive disorder, or manic depression) is meant to encompass any type and severity of mood disorder, wherein an individual suffering experiences episodes of a frenzied state known as mania, typically alternating with episodes of depression.

The term “neuropathic pain” is meant to encompass any type, frequency (continuous and/or episodic (paroxysmal)) and severity of pain caused by damage or disease that affects the somatosensory system. This term includes also any disorders or conditions associated therewith including, but not limited to abnormal sensations called dysesthesia, pain produced by normally non-painful stimuli (allodynia), burning or coldness sensations, “pins and needles” sensations, numbness and itching, nociceptive pain.

The term “fibromyalgia” is meant to encompass any type and severity of chronic widespread pain and allodynia (a heightened and painful response to pressure) that may be caused by any psychological, genetic, neurobiological or environmental factors. The term includes any conditions and disorders associated therewith including, but not limited to pain, debilitating fatigue, sleep disturbance, and joint stiffness, difficulty with swallowing, bowel and bladder abnormalities, numbness and tingling, muscle and connective tissue pain.

The term “conditions associated menopause” is meant to encompass any unwanted and unpleasant conditions associated with menopause hormonal change, specifically the decrease in estrogen. Such conditions include, but are not limited to hot flashes night sweats, menstrual irregularities, vaginal dryness, migraines, insomnia, anxiety, irritability, mood changes, depression.

The diseases and disorders listed herein, including anxiety, depression (including post partum depression) conditions associated menopause, stress, bipolar disorder and fibromyalgia belong to a class of diseases and disorders associated with affective disorders that are related to mood and pain disorders, however do not relate to cognitive function or obesity.

In some embodiments the compounds as disclosed in the present invention are used in a method of treating a disease, disorder or condition associated with affective disorder.

As used herein, the phrase “affective disorders” collectively describes any psychological and/or psychotic disorder characterized by an undesirable excess of emotions such as, but not limited to, sadness, fear, anxiety, and encompasses any disorder that may be treated effectively by essentially the same treatments as the aforementioned disorders.

Non-limiting examples of affective disorders include depressive disorders, bipolar disorders (e.g., manic depression), cataplexy, cyclothymia, dysthymia, anxiety disorders, panic disorders, phobias (including social phobias) and premenstrual dysphoric disorder.

As used herein, the phrase “depressive disorders” describes affective disorders that are characterized by depression as a symptom. Examples of depressive disorders include, without limitation, clinical depression, dysthymia, bipolar disorders (e.g., manic depression) and cyclothymia.

The term “for treatment of conditions that benefit from raise in leptin or POMC levels” refers to diseases that show an improved clinical outcome wherein leptin or POMC levels are raised whether initially there were abnormally low, or normal levels of these hormones.

Leptin is an adipocyte-derived hormone with antidepressant-like properties was shown to restore adult hippocampal neurogenesis suppressed by chronic unpredictable stress and reverses glucocorticoid induced inhibition of GSK3β/β-catenin signaling. Stress and glucocorticoid stress hormones inhibit neurogenesis, whereas antidepressants increase neurogenesis and block stress-induced decrease of neurogenesis. Chronic treatment with leptin reversed the CUS-induced reduction of hippocampal neurogenesis and depression-like behaviors. Leptin treatment elicited delayed long-lasting antidepressant-like effects in the behavioral despair test (Garza J C et al. Mol. Psychiatry, 17(8), 790-808 (2012)).

POMC is the precursor of beta-endorphin. The pathways for stress-related psychiatric disorders, depression and PTSD, converge to a common pathway in which beta-endorphin is a modulating element of distress. This may occur via its interaction with the mesolimbic monoaminergic system and also by its effects on learning and memory. Curr Drug Targets. 2009 November; 10(11): 1096-108.

Conditions where leptin is especially beneficial are connected to survival such as in cases on malnutrition, stress, anorexia nervosa, cachexia, depression, sepsis, depression. Conditions where enhancement of POMC are beneficial are obesity and adrenal insufficiency, morphine and heroin addiction, chronic pain, PTSD, depression, steroidogenesis, energy homeostasis, melanocyte stimulation, immune modulation, production of melanin) lipolysis and steroidogenesis. Problems in circadian rhythm appetite and sexual arousal photoprotection in patients with erythropoietic protoporphyria, polymorphous light eruption, actinic keratosis, squamous cell carcinoma (a form of skin cancer), enhanced libido.

The term “treatment” as used herein refers to the administering of a therapeutic amount of a compound or a composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The “effective amount” for purposes disclosed herein is determined by such considerations as may be known in the art. The amount must be effective to achieve the desired therapeutic effect as described above, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half life in the body, on undesired side effects, if any, on factors such as age and gender, etc.

Compounds as disclosed herein above may be formulated to pharmaceutical compositions. Such composition may comprise additionally any other suitable substances such as other therapeutically useful substances, diagnostically useful substances, pharmaceutically acceptable carriers or the like.

The present invention thus also relates to pharmaceutical compositions comprising a compound disclosed in the subject invention in admixture with pharmaceutically acceptable auxiliaries, and optionally other therapeutic agents. The auxiliaries must be “acceptable” in the sense of being compatible with the other ingredients of the composition and not deleterious to the recipients thereof.

Pharmaceutical compositions include those suitable for oral, rectal, nasal, topical (including transdermal, buccal and sublingual), vaginal or parenteral (including subcutaneous, intramuscular, intravenous and intradermal) administration or administration via an implant. The compositions may be prepared by any method well known in the art of pharmacy.

Such methods include the step of bringing in association compounds used in the invention or combinations thereof with any auxiliary agent. The auxiliary agent(s), also named accessory ingredient(s), include those conventional in the art, such as carriers, fillers, binders, diluents, disintegrants, lubricants, colorants, flavouring agents, anti-oxidants, and wetting agents.

Pharmaceutical compositions suitable for oral administration may be presented as discrete dosage units such as pills, tablets, dragées or capsules, or as a powder or granules, or as a solution or suspension. The active ingredient may also be presented as a bolus or paste. The compositions can further be processed into a suppository or enema for rectal administration.

The invention further includes a pharmaceutical composition, as hereinbefore described, in combination with packaging material, including instructions for the use of the composition for a use as hereinbefore described.

For parenteral administration, suitable compositions include aqueous and non-aqueous sterile injection. The compositions may be presented in unit-dose or multi-dose containers, for example sealed vials and ampoules, and may be stored in a freeze-dried (lyophilised) condition requiring only the addition of sterile liquid carrier, for example water, prior to use. For transdermal administration, e.g. gels, patches or sprays can be contemplated. Compositions or formulations suitable for pulmonary administration e.g. by nasal inhalation include fine dusts or mists which may be generated by means of metered dose pressurized aerosols, nebulisers or insufflators.

The exact dose and regimen of administration of the composition will necessarily be dependent upon the therapeutic or nutritional effect to be achieved and may vary with the particular formula, the route of administration, and the age and condition of the individual subject to whom the composition is to be administered.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 shows the 5HT levels of compounds of the invention vs. control in the hippocampus.

FIG. 2 shows the norepinephrine levels of compounds of the invention vs. control in the hypothalamus.

FIG. 3 shows the 5HT levels of compounds of the invention vs. control in the hypothalamus.

FIG. 4 shows the norepinephrine levels of compounds of the invention vs. control in the hippocampus.

FIG. 5 shows the weight loss in Sabra mice affected by compounds X, 12 and 15.

FIG. 6 shows the weight loss in OB/OB mice affected by compounds X, 12 and 15.

FIG. 7 shows the results of Tail Suspension Test of compounds X, 12 and 15 as compared with imipramine.

FIG. 8 shows the results of the FST of compounds X, 12 and 15 as compared with imipramine.

FIGS. 9A-9C show the results of the NSFT of compounds X, 12 and 15 as compared with impramine.

FIG. 10 shows the results of the eight arm maze test of compounds X, 12 and 15 as compared with imipramine.

FIG. 11 shows POMC protein expression in the hypothalamus following OVA (oleyl-L-valinol amide) administration (p<0.001a).

FIG. 12 shows POMC protein expression in the hippocampus following OVA (oleyl-L-valinol amide) administration (p<0.001a).

FIG. 13 shows NPY protein expression in the hypothalamus following OVA (oleyl-L-valinol amide) administration (p<0.001a).

FIGS. 14A-14B shows AMPK protein expression in the hippocampus (14A) and hypothalamus (14B) following OVA (oleyl-L-valinol amide) administration (p<0.001a).

FIG. 15 shows leptin levels in the hypothalamus following OVA (oleyl-L-valinol amide) administration (p<0.001a).

FIG. 16 shows p-SAPK/JNK expression in the hypothalamus following OVA (oleyl-L-valinol amide) administration (p<0.001a).

FIG. 17 shows the results of Forced Swim Test of compound X (0.1, 1, 10 mg/Kg) as compared with imipramine.

FIG. 18 shows the results of Tail Suspension Test of compound X (0.1, 1, 10 mg/Kg) as compared with imipramine.

FIG. 19 shows the results of Open Field Test of compound X (0.1, 1, 10 mg/Kg) as compared with imipramine.

### DETAILED DESCRIPTION OF EMBODIMENTS

### Example 1

### Norepinephrine (NE) and Serotonin (5HT) Determination

Compounds

Serotonin was purchased from Sigma cat.NH-7752,

L-Norepinephrine was purchased from Fluka (cat.N 74480)

3,4-dihydroxybenzylamine (DHBA) was purchased from Sigma cat.Nd-7012, all the other chemicals used were obtained from Sigma.

Working Solutions

Solution of DHBA as internal standard in the concentration of: 1 μg/ml in 0.1M perchloric acid, were prepared. Standard solutions of NE and 5HT in the concentration of 1-0.1 μg/ml in 0.1M perchloric acid were prepared.

Sample Preparation

Tissues (hypothalamus and hippocampus) obtained from 6-8 mice were dissected on ice surface and kept at −80 C. Hypothalamus (HT) or hippocampus (HC) from 1 mice were weighted (weight of HT −10-20 mg, HC 30-40 mg) and placed in eppendorf micro-centrifuge tube, 1.5 ml. To each tube was added 120-160 μl of ice cold solution of Perchloric acid and DHBA, homogenized in ice bath on Sonics vibra cell, 3×20 sec., 0.4 watts and centrifuged at 14,000×g at 4° C. 10 min. Supernatant gained was stored in ice before analysis.

Chromatography

The HPLC system consisted of Jasco PU-980 pump, Jasco AS-950-10 autosampler cooled at 4° C., H3 ODS-125A 3 μm 125 mm×4.6 mm i.d. analytical column (Hichrom, UK) were used. Electrochemical detector Coulochem ESA 5100A with analytical cell model 5011 was used for control detector voltage and record the current. Working electrode was set at potential +350 mV. Control and data collection/processing were handled through Borwin chromatography software.

The mobile phase composition: A stock buffer was prepared as follows: 20 mM sodium dihydrogenorthophosphate, EDTA 3 mg/l, 1-heptanesulfonic acid 1 g/l were dissolved in 1 liter deionized distilled water and buffered to pH 3.7 using concentrated phosphoric acid. To prepare the mobile phase buffer was mixed with acetonitrile (Baker HPLC analyzed) in the ratio 85:15 (v/v), filtered through a 0.2 μm membrane filter and degassed under vacuum after mixing. Mobile phase flow rate 1 ml/min. Injection volume 20 μl, retention time for DHBA 4.2 min., norepinephrine—3.1 min., 5HT 8.6 min. For calibration curve 1 ug/ml solution of DHBA together with 1.0-0.1 μg/ml solutions of 5HT and NE were used. The ratio between 5HT or NE and DHBA used to calculate the level of 5HT and NE in the sample. Date are given in ng/g wet tissue +/−SEM.

Results

oleyl-L-valinol (compound “1”), oleyl-amine-carbonyl-D-valinol (compound “14”), and oleyl-amine-carbonyl-L-valinol (compound “15”) significantly enhanced both Norepinephrine (NE) and serotonin (5HT) levels in the hypothalamus and hippocampus. Hippocampus 5-HT compound 1 (p<0.01), compound 14 (p<0.01), compound 15 (p<0.001). Hippocampus NE compound 1 (p<0.01), compound 14 (p<0.01), compound 15 (p<0.001). Hypothalamus 5-HT compound 1 (p<0.01), compound 14 (p<0.05), compound 15 (p<0.05). Hypothalamus NE compound 1 (p<0.05), compound 14 (p<0.05) compound 15 (p<0.05).

Compounds 4, 10, 12, 13, 19 significantly enhanced both Norepinephrine (NE) and serotonin (5HT) levels in the hippocampus as follows: 4 (p<0.001) for 5-HT and (p<0.05) for NE, 10 for 5-HT (p<0.001) and NE (p<0.001), 12 for 5-HT (p<0.01) and NE (p<0.001), 13 for 5HT (p<0.01) and NE (p<0.001) and for 19, 5HT ((p<0.01) and NE (p<0.05).

Tables 1 and 2 show the results of compounds of the invention in the hypothalamus and hippocampus.

TABLE 1
Hypothalamus

| com- | | | | | | |
|---|---|---|---|---|---|---|
| pound | 5HT (ng/g) | t-test | n | NE (ng/g) | t-test | n |
| control | 704.3 +/− 9.3 | | 10 | 1009.5 +/− 20.4 | | 23 |
| comp. 1 | 854.4 +/− 16.8 | <0.001 | 16 | 1090 +/− 10.4 | <0.05 | 22 |
| comp. 5 | 767.2 +/− 51.9 | <0.05 | 4 | | | |
| comp. 7 | 856.5 +/− 115.2 | <0.01 | 4 | | | |
| comp. | 777.8 +/− 79.1 | <0.01 | 5 | | | |
| 11 | | | | | | |
| comp. | 810.5 +/− 70.6 | <0.01 | 13 | | | |
| 12 | | | | | | |
| comp. | 831.3 +/− 29.1 | <0.01 | 4 | 1002 +/− 62.7 | >0.05 | 5 |
| 13 | | | | | | |
| comp. | 876.4 +/− 126.8 | <0.05 | 4 | 1139.4 +/− 43.3 | <0.05 | 4 |
| 14 | | | | | | |
| comp. | 853 +/− 78.3 | <0.05 | 4 | 1234.8 +/− 59.8 | <0.01 | 4 |
| 15 | | | | | | |

TABLE 2
Hippocampus

| com- | | | | | | |
|---|---|---|---|---|---|---|
| pound | 5HT (ng/g) | t-test | n | NE (ng/g) | t-test | n |
| control | 253.1 +/− 5.7 | | 36 | 215.1 +/− 13 | | 46 |
| comp. 1 | 342.2 +/− 33 | <0.001 | 27 | 283.28 +/− 24 | <0.01 | 27 |
| comp. 4 | 380.4 +/− 12.8 | <0.001 | 4 | 302.5 +/− 22.4 | <0.05 | 4 |
| comp. 5 | 419.8 +/− 67.4 | <0.0001 | 5 | | | |
| comp. 7 | 335.4 +/− 45.4 | <0.01 | 3 | | | |
| comp. | 299.2 +/− 22.8 | <0.001 | 5 | 407.9 +/− 48.9 | <0.001 | 5 |
| 10 | | | | | | |
| comp. | 332.1 +/− 52.7 | <0.01 | 3 | 378.9 +/− 31.4 | <0.001 | 4 |
| 12 | | | | | | |
| comp. | 300.1 +/− 41.5 | <0.01 | 4 | 396.7 +/− 44.7 | <0.001 | 3 |
| 13 | | | | | | |
| comp. | 344 +/− 32.4 | <0.001 | 4 | 313.9 +/− 48 | <0.01 | 4 |
| 14 | | | | | | |
| comp. | 396.7 +/− 21 | <0.0001 | 4 | 373.7 +/− 48.1 | <0.001 | 4 |
| 15 | | | | | | |
| comp. | 296.3 +/− 33.2 | <0.01 | 5 | 296.4 +/− 24.8 | <0.05 | 4 |
| 19 | | | | | | |
| comp. | | | | 242.1 +.− 13.5 | <0.05 | 6 |
| 26 | | | | | | |

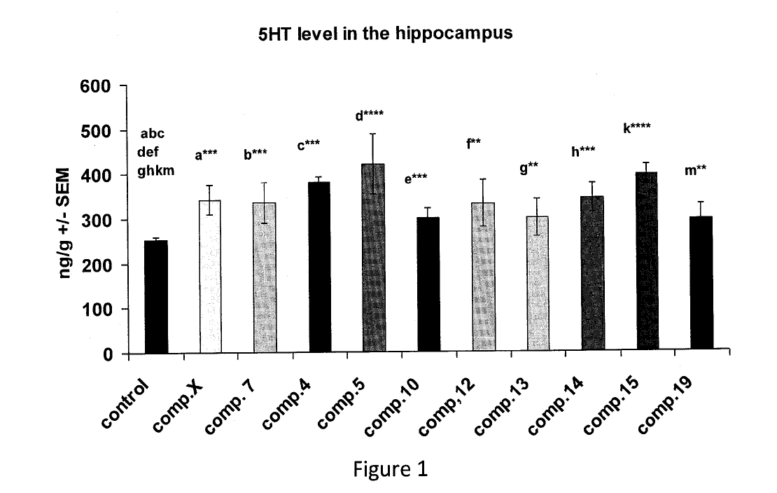

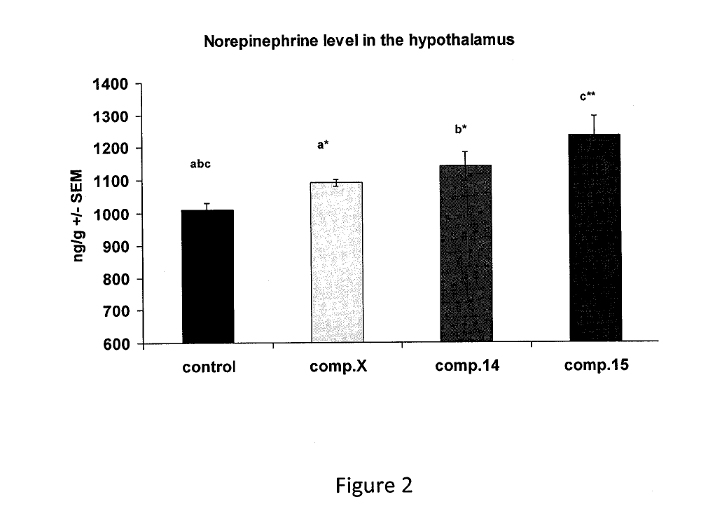

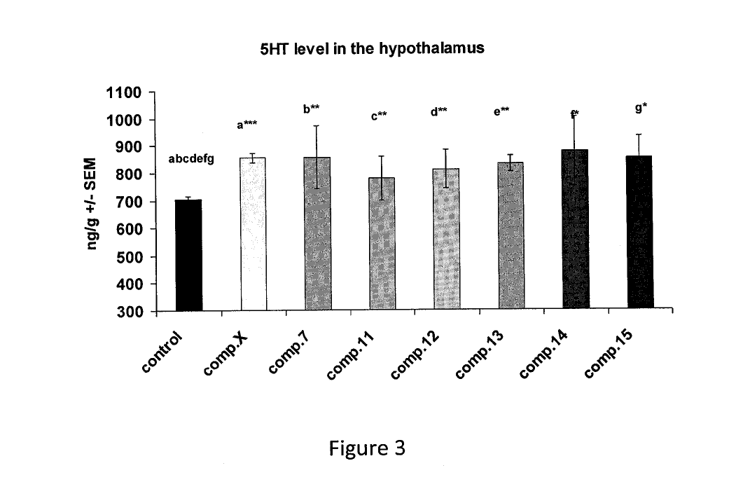

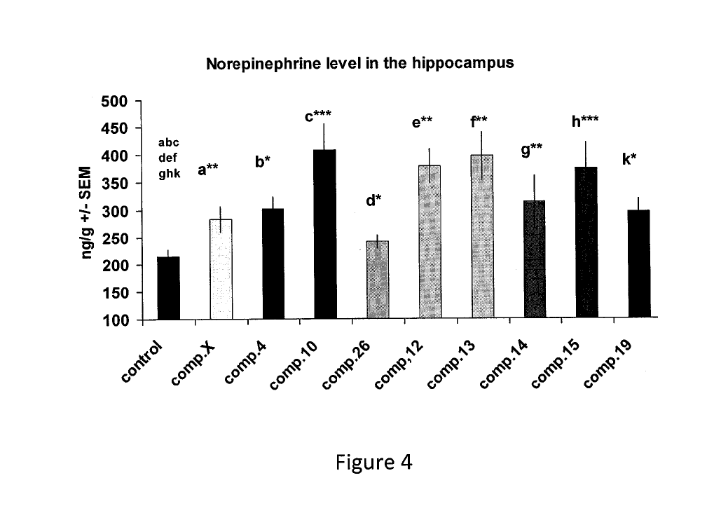

FIG. 1 shows that 5HT levels in the hippocampus increased significantly following administration of compounds X, 7, 4, 5, 10, 12, 13, 14, 15, 19. FIG. 2 shows norepinephrine levels in the hypothalamus increased significantly following administration of compound X, 14, 15. FIG. 3 shows 5HT levels in the hypothalamus increased significantly following compounds X, 7, 11, 12, 13, 14, 15 administration. FIG. 4 shows norepinephrine levels in the hippocampus increase significantly following compounds X, 4, 10, 26, 12, 13, 14, 15 and 19 administration.

### Example 2

### In Vivo Sabra Mice Experiment

Paradigms that employ acute or subchronic stress exposure include: forced swim test (FST), Novelty suppressed feeding test (NSFT) and tail suspension test (TST), which employ relatively short-term exposure to inescapable or uncontrollable stress and can reliably detect antidepressant drug response

Behavioral Tests

140 Female Sabra Mice 12 weeks old are divided to 13 experimental groups 10 mice in each group with equal weight. Mice are administered I.P. with either saline or 1 mg/kg Fluoxetine (PROZAC), compound 1, 4, 5, 7, 10, 11, 12, 13, 14, 15, 19, 26 for a 4 weeks period. On day 30 and on, mice were tested by the following behavioral tests.

The temporal sequence of the behavioral tests is adopted relative to the putative stressogenic effect of each test (from low to high: NSFT, TST, FST) to avoid possible carryover effects (Zhang J, Lazar M A. Annu Rev Physiol. 62:439-66(2000)).

Novelty suppressed feeding test (NSFT): After the last treatment injection, all food is removed from the cage for 24 hours (water available ad libitum). At the end of this time, the animal is introduced into a 50×50×20 (height) cm wooden arena the floor of which is marked with equal rectangles of 10×10 cm. A pellet of food is placed on an elevated surface in the center of the arena. The time elapsing from the introduction of the animal into the arena until it commences eating (latency to feed) is recorded. The animal is removed from the arena immediately after it begins to eat or after not doing so for 5 min. During the test, the number of lines crossed by the animal is counted as a measure of motor activity, and calibrated for 5 min if the animal started eating before that. After the test, the animal is immediately transferred to its home cage and left to consume a previously weighed amount of food for 10 minutes. On completion of this period the food is weighed again to calculate the home cage food consumption.

The rating of the animals' behavior in each of the above paradigms is conducted by two experimenters who are blind to the treatment received by each mouse. The mean of the two ratings is calculated and used for the statistical analysis.

Tail suspension test (TST): Animals are suspended upside down by adhesive tape placed 1 cm from the tail tip. The elevation of the animal is 50 cm from the nearest surface. The test duration is 6 minutes during which the animal's behavior is rated every 5 seconds as “active” or “inactive”. Statistical analysis compares the magnitude of immobility (in seconds) between different treatment groups. In this test, antidepressants characteristically cause an increase of the time fraction spent by the animal as active at the expense of the inactivity period.

Forced swim test (FST): The animal is placed in a circular, transparent, plexiglass tank measuring 21 cm in diameter and 46 cm in height containing water 15 cm high, maintained at 23-25° C. During a 6 minutes test period, the activity is rated every five seconds as either “active” (swimming) or “inactive” (immobile, performing only movements to keep itself from drowning). Also measured is the latency period, the time elapsing, for each animal from immersion in the water tank until the first occurrence of immobility. Since climbing activity is minimal in mice, this variable is included in the statistical analysis.

Characteristically, antidepressant treatment causes an increase of the time fraction spent by the animal in activity at the expense of immobility out of the total test duration. After completion of the test, the animals are towel dried and placed for 15 min near a heating device before being returned to their home cage. The mice is followed for survival during the experiment. Mice are sacrificed after 40 days, brains are tested for brain catecholamines and serotonin by HPLC-ECD and for brain BDNF and SIRT gene expression (both enhanced expression of BDNF and SIRT are associated with depression). The analysis is performed on brain areas associated with depression such as Medial prefrontal cortex (MPFC), amygdale and hippocampus.

Measurement of Monoamines

Mice were sacrificed by decapitation on the 40th day. Medial prefrontal cortex (MPFC), amygdale and hippocampus are immediately dissected out and kept at −70° C. for all measurements. Assay for NE, dopamine and 5-HT w was performed by high-performance liquid chromatography/electrochemical detector using the same procedure reported in behavioral and neurochemical alterations caused by diet restriction—the effect of tyrosine administration in mice.

Quantitative Reverse Transcription—Polymerase Chain Reaction Analysis

Mice are sacrificed by decapitation on the 12th day and total Medial prefrontal cortex (MPFC), amygdale and hippocampus RNAs are extracted using Tri reagent according to the manufacturer's instructions and are reverse transcribed. RNA samples with no reverse transcription (RT) are amplified in the polymerase chain reaction (PCR) in order to rule out the possibility of amplifying genomic DNA contamination which was present in the RNA extracted from the tissue.

Quantitative RT-PCR was carried out with Power SYBR Green PCR Master Mix (Applied Biosystems, UK), in 7900HT instrument (Applied Biosystems). Volume reaction is 15 μl and glyceraldehyde 3-phosphate dehydrogenase (GAPDH) is used as endogenous control. Threshold cycle (Ct) is determined by SDS software for each one of the samples tested, and the average Ct is calculated for each triplicate. ΔCt of each target gene is calculated by subtracting the average Ct for GAPDH of a given sample from the average Ct for the target gene of the same sample. Average ΔCt of a certain target gene in the control group was subtracted from ΔCt of the same gene in samples from the treated groups to yield ΔΔCt of this gene in the sample. The quantity of a specific target gene in a certain sample relative to the control group w was calculated as 2−ΔΔCt utilizing ΔΔCt determined for that sample. All primers were synthesized by Syntezza (Jerusalem, Israel)

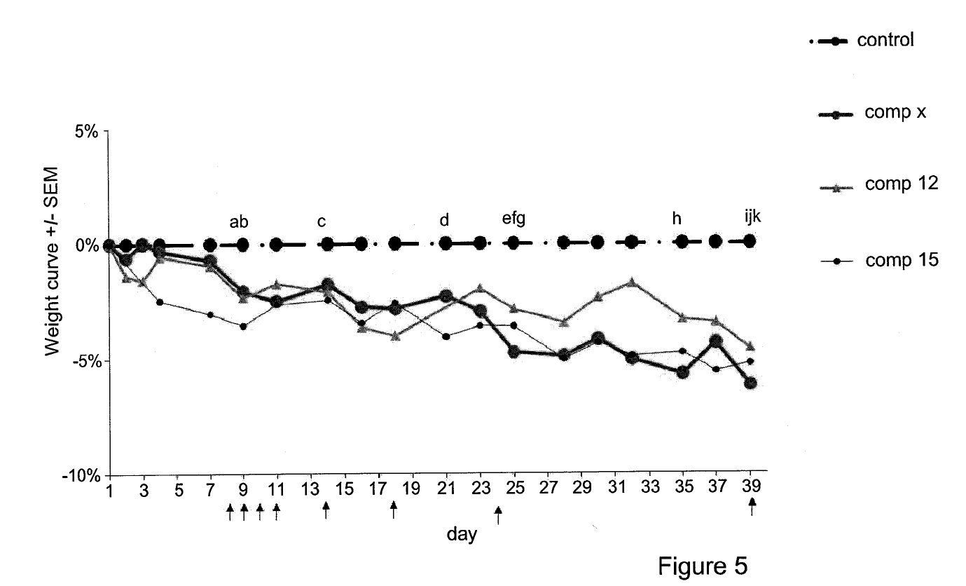

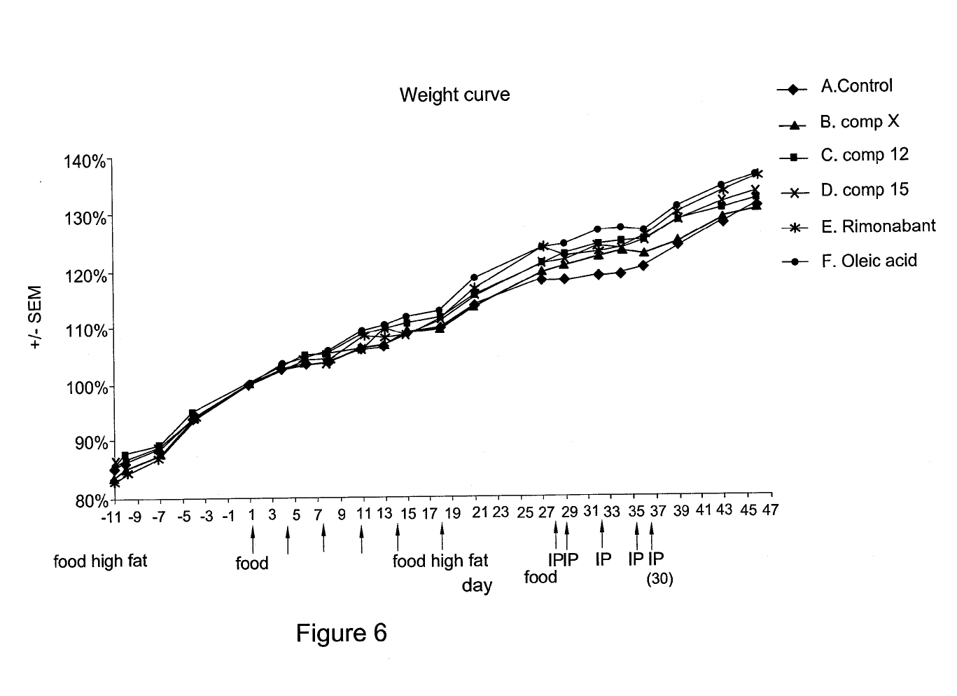

FIG. 5 shows that compounds X, 12 and 15 produce significant weight loss in Sabra mice. FIG. 6 shows that Compounds X, 12 and 15 do not produce weight loss in ob/ob mice. Injection of the mentioned compounds to Sabra mice caused significant decrease in food consumption and weight however administration of the same compounds to OB/OB mice which lack the leptin gene did not change either food consumption or weight. Thus the compounds of the invention can be used to treat conditions that are mediated by the enhancement of leptin levels. In obesity there is high leptin level however the leptin receptors are not responding. High CNS leptin level has anti-depression effect however, if there is tolerance there is no anti depression effect. In the article we have shown that OVA decreased body weight of both Sabra and C57BL/6 mice but not body weight of OB/OB mice that lack the leptin gene it seems that OVA is mediated by leptin.

### Example 3

### In Vivo BALB/c Experiment

The BALB/c mice were chosen as a model of depression. BALB/c mice show reduced locomotor activity, less time in unprotected and brightly lit areas in the open field and light: dark assays, stress-induced increases in corticosterone release and ACTH as well as basal differences in CRH, and stress-induced differences in CRH receptor immunoreactivity, Basal and stress-induced differences in expression of GABA A receptor subunits.

Experimental Procedures

Active Compounds:

Oleoyl-L-Valinol (compound X), Oleyloxycarbonyl-L-Valinolamide (compound 12), and Oleoylaminocarbonyl-L-Valinol ureide (compound 15) in comparison to imipramine in the treatment of metabolic syndrome associated depression.

Mice administered with the above compounds were tested in the Tail Suspension Test, Forced Swim Test and Novel Suppressed Feeding Test—gold standard tests for evaluation of anti-depressive activity.

Tail Suspension Test

50, 2 month old female BALB/c mice were separated (each mouse in a different cage) for 9 days period (a model for depression). On days 7, 8, 9, 34-40 mice were administered with either vehicle or compounds X, 12 and 15 (1 mg/kg i.p.) or imipramine 10 mg/kg (anti-depressant) i.p. and tested in the Tail suspension test on day 7 of the experiment (10 mice per group).

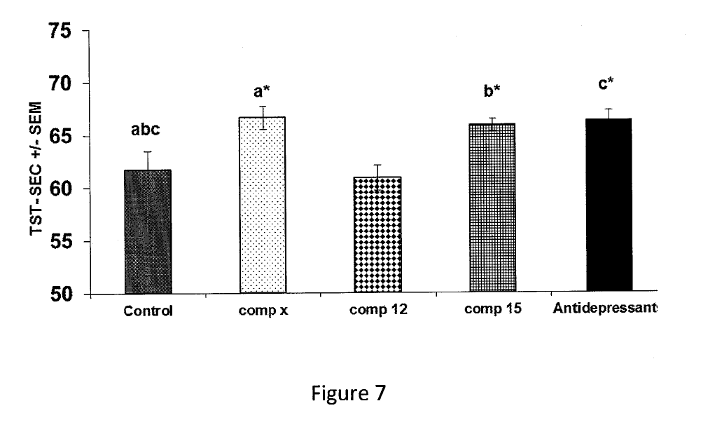

FIG. 7 shows the results of Tail Suspension Test. Compounds X and 15 as well as imipramine showed significantly enhanced activity in relation to vehicle and imipramine (1 mg/kg of compounds in relation to 10 mg/kg imipramine).

Forced Swim Test (FST)

50, 2 month old female BALB/c mice, 10 mice per group, on ad libitum diet were separated (each mouse in a different cage) for 9 days (Separation Stress, a model for depression). On the 7, 8, 9, 34-40 days mice were administered with either vehicle or compounds x, 12 and 15 (1 mg/kg i.p.) or imipramine 10 mg/kg (antidepressant) i.p. and tested in the forced swim test (FST) on day 8.

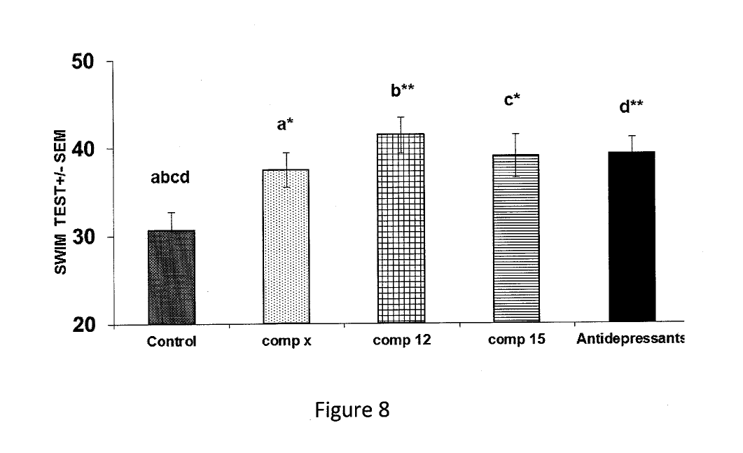

FIG. 8 shows the results of the FST. Compounds X, 12 and 15 as well as imipramine showed significantly enhanced swimming activity in relation to vehicle. The enhanced swimming activity with the novel compounds (1 mg/kg) resembled that of imipramine (10 mg/kg), a well known antidepressant.

Novelty Suppressed Feeding Test (NSFT)

50, 2 month old female BALB/c mice, 10 mice per group, on ad libitum diet were separated (each mouse in a different cage) for 9 days (Separation Stress, a model for depression). On the 7, 8, 9, 34-40, days mice were administered with either vehicle or compounds x, 12 and 15 (1 mg/kg i.p.) or imipramine 10 mg/kg (antidepressant) i.p. and tested in Novel Suppressed Feeding Test on day 9.

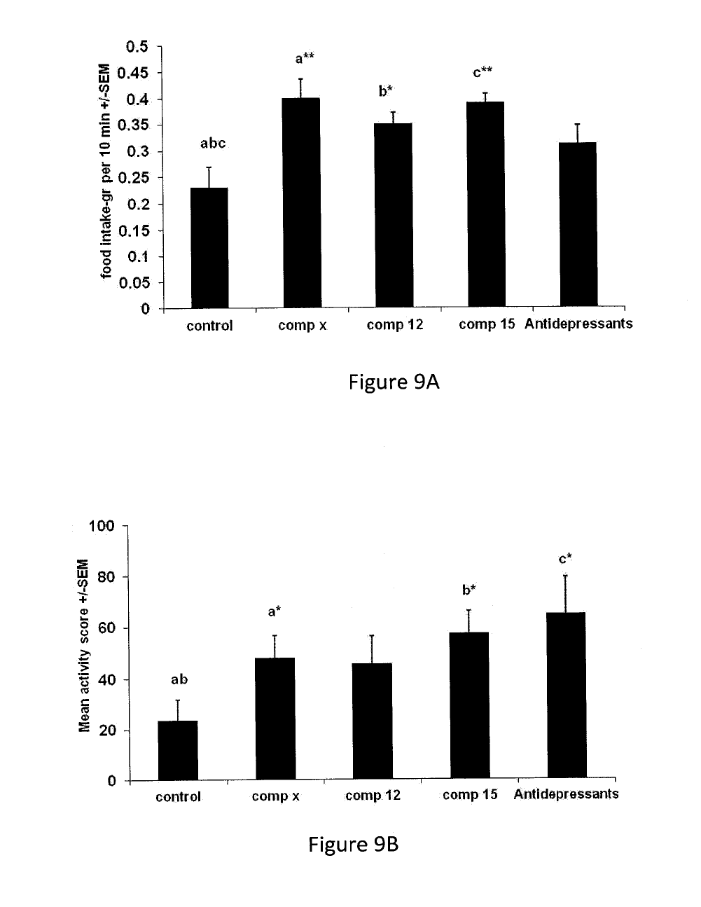

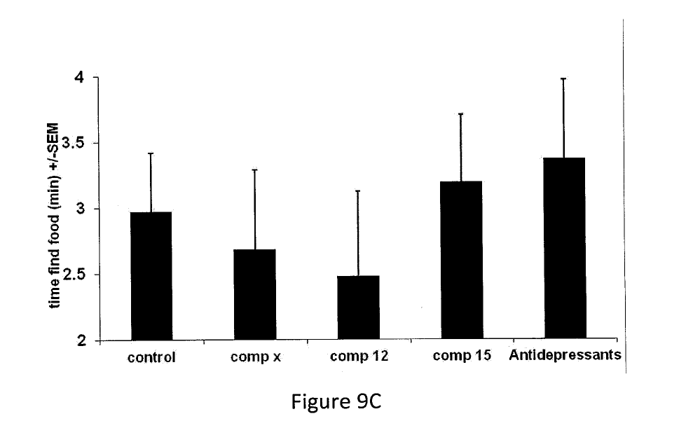

FIGS. 9A-9C show the results of the NSFT. Compounds X, 12 and 15 showed significantly enhanced food intake in relation to vehicle and more than impramine. The enhanced motor activity with the novel compounds (1 mg/kg) resembled that of imipramine (10 mg/kg), a well known antidepressant. After the last treatment injection, all food is removed from the cage for 24 hours. At the end of this time, the animal is introduced into the open field test. A pellet of food is placed on an elevated surface in the center of the arena. The time to find the food was recorded. The animal is removed from the arena immediately after it begins to eat or after not doing so for 5 min. During the test, the number of lines crossed by the animal is counted as a measure of motor activity, and calibrated for 5 min if the animal started eating before that. After the test, the animal is immediately transferred to its home cage and left to consume a previously weighed amount of food for 10 minutes. On completion of this period the food is weighed again to calculate the home cage food consumption.

Eight Arm Maze Test

2 month old female BALB/c mice 10 mice per group were separated (each mouse in a different cage) for 9 days (a model for depression). On days 7, 8, 9, 34-40 days mice were administered with either vehicle or compounds X, 12 and 15 (1 mg/kg i.p.) or imipramine 10 mg/kg (anti-depressant) i.p. and tested in the Eight Arm Maze from day 34-38.

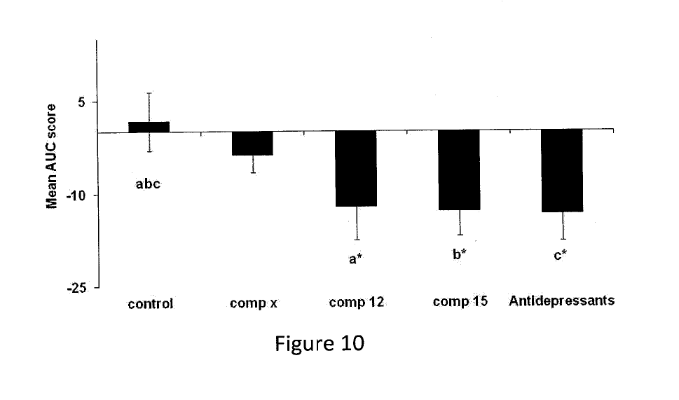

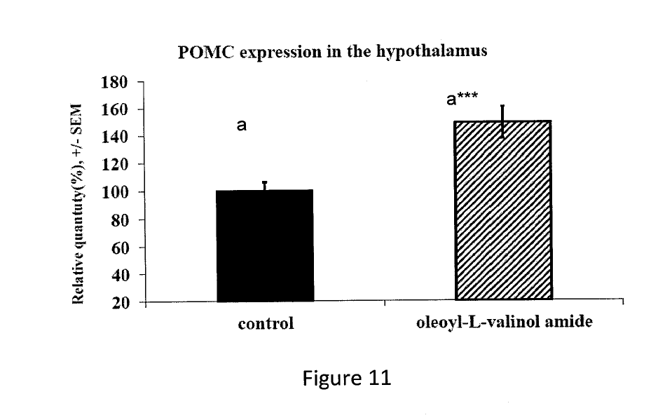

FIG. 10 shows the results of the eight arm maze test. Compounds 12 and 15 as well as imipramine showed significantly improved performance in the Eight Arm Maze in relation to vehicle (p<0.05). Thus they improved also cognition and did not affect neurological score as measured by the NSS method.

POMC Expression

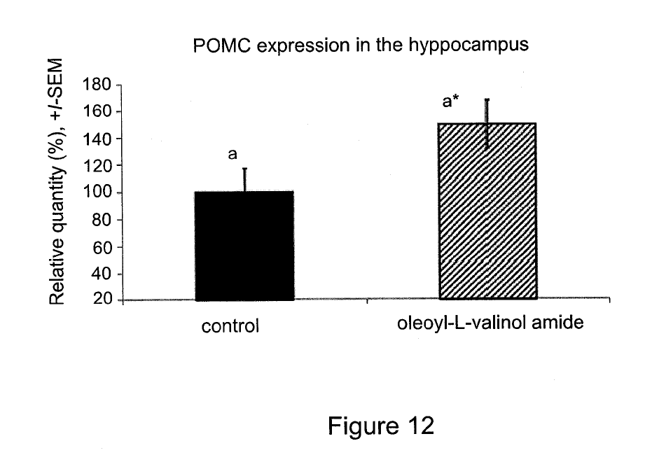

50, 2 month old female BALB/c mice, 10 mice per group, on ad libitum diet were separated (each mouse in a different cage) for 9 days (Separation Stress, a model for depression). On the 7, 8, 9, 34-40 days mice were administered with either vehicle or compounds x, 12 and 15 (1 mg/kg i.p.) or imipramine 10 mg/kg (antidepressant). POMC protein was monitored by western blot. The results presented in FIG. 11 and FIG. 12 show that POMC protein increased significantly following OVA administration (p<0.001a).

NYP Expression

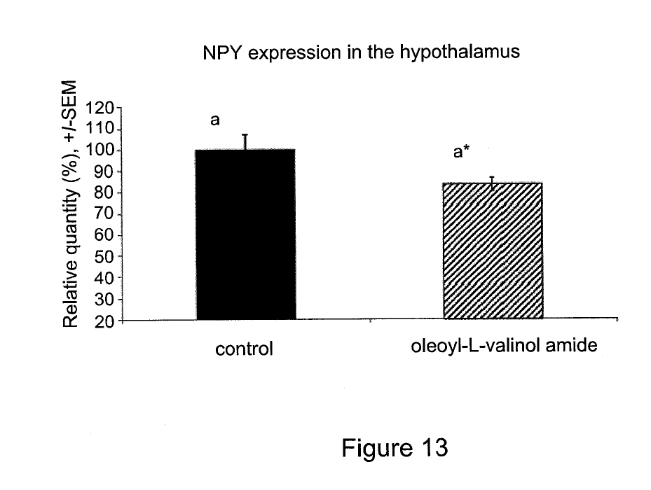

50, 2 month old female BALB/c mice, 10 mice per group, on ad libitum diet were separated (each mouse in a different cage) for 9 days (Separation Stress, a model for depression). On the 7, 8, 9, 34-40 days mice were administered with either vehicle or compounds X, 12 and 15 (1 mg/kg i.p.) or imipramine 10 mg/kg (antidepressant). The results shown in FIG. 13 demonstrate that NPY protein was monitored by western blot. NPY decrease significantly following OVA administration (p<0.05a).

P-AMPK Levels

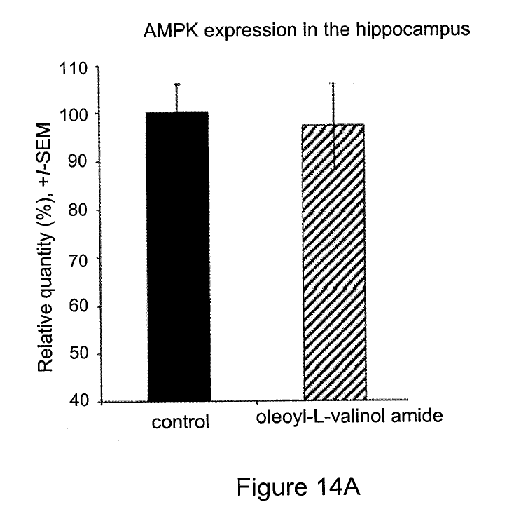

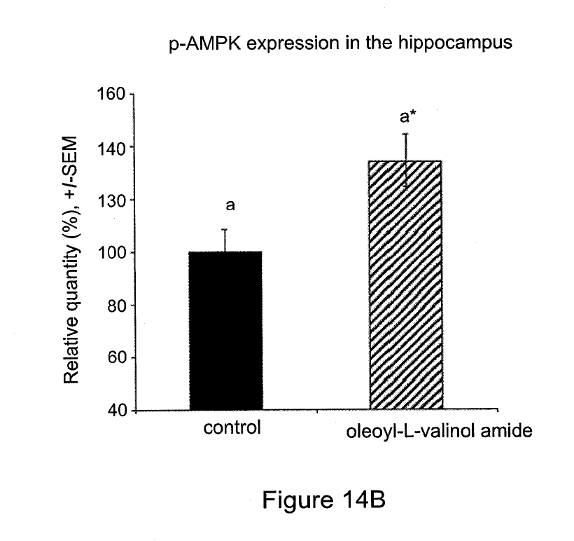

50, 2 month old female BALB/c mice, 10 mice per group, on ad libitum diet were separated (each mouse in a different cage) for 9 days (Separation Stress, a model for depression). On the 7, 8, 9, 34-40 days mice were administered with either vehicle or compounds x, 12 and 15 (1 mg/kg i.p.) or imipramine 10 mg/kg (antidepressant). P-AMPK protein was monitored by western blot. FIGS. 14A and 14B shows that there was significant increase in P-AMPK following OVA administration. (p<0.05a).

Leptin Levels

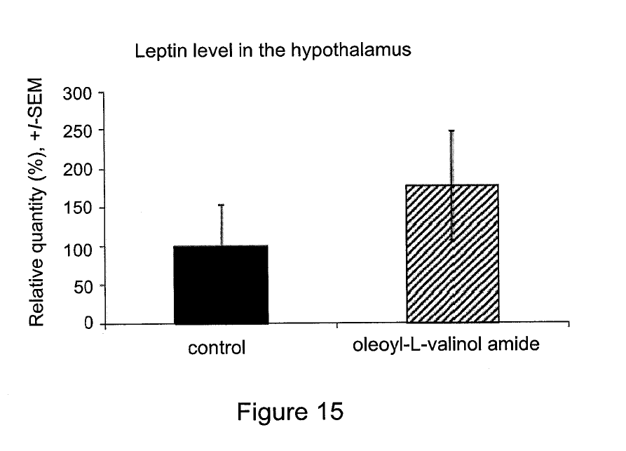

50, 2 month old female BALB/c mice, 10 mice per group, on ad libitum diet were separated (each mouse in a different cage) for 9 days (Separation Stress, a model for depression). On the 7, 8, 9, 34-40 days mice were administered with either vehicle or compounds x, 12 and 15 (1 mg/kg i.p.) or imipramine 10 mg/kg (antidepressant) FIG. 15 shows that there was tendency to increase in leptin levels following OVA administration.

p-SAPK/JNK Levels

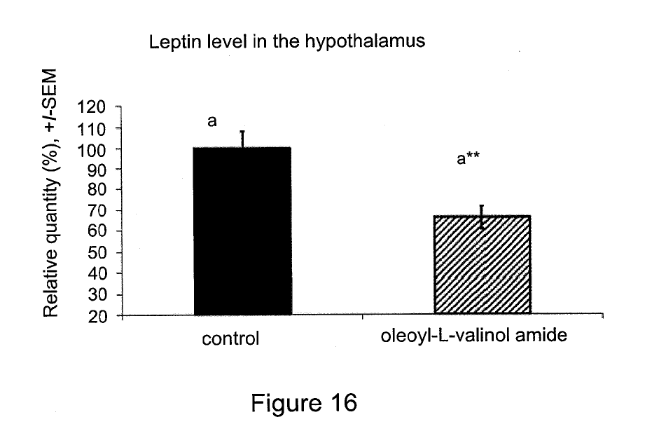

50, 2 month old female BALB/c mice, 10 mice per group, on ad libitum diet were separated (each mouse in a different cage) for 9 days (Separation Stress, a model for depression). On the 7, 8, 9, 34-40 days mice were administered with either vehicle or compounds x, 12 and 15 (1 mg/kg i.p.) or imipramine 10 mg/kg (antidepressant) FIG. 16 shows that there was significant decrease in p-SAPK/JNK levels following OVA administration (p<0.01).

Forced Swim Test

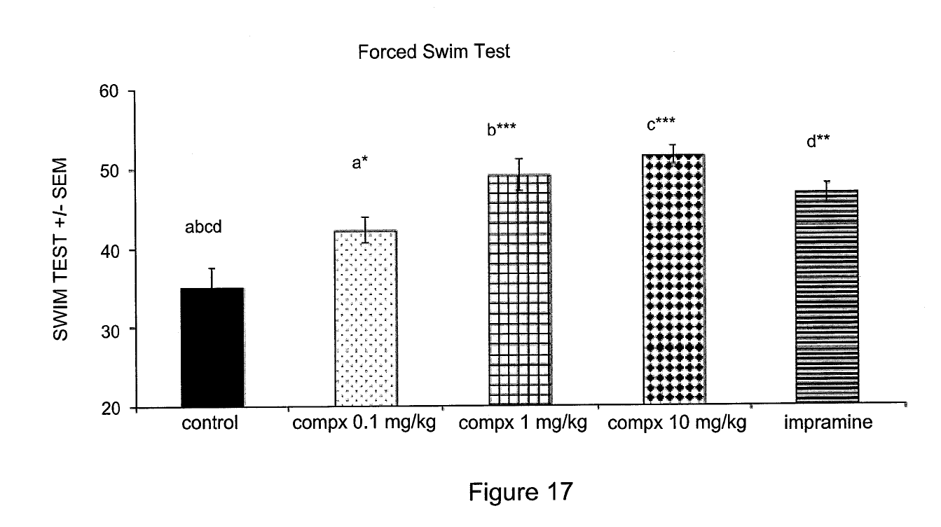

50, 2 month old BALB/c mice, 10 mice per group, on ad libitum diet were divided to 5 experimental groups with equal weight. On the 2, 3, 4, 5, 9, 11, 16, 18, 23, 25, 29, 31 days mice were administered with either vehicle or compound X 0.1, 1, 10 mg/kg i.p. or imipramine 10 mg/kg. Forced Swim Test was evaluated on day 11. FIG. 17 shows that 0.1, 1 and 10 mg/kg of Compound X improved significantly swimming activity (p<0.05a, p<0.01 bc) 1 and 10 mg/kg better than impramine.

Tail Suspension Test

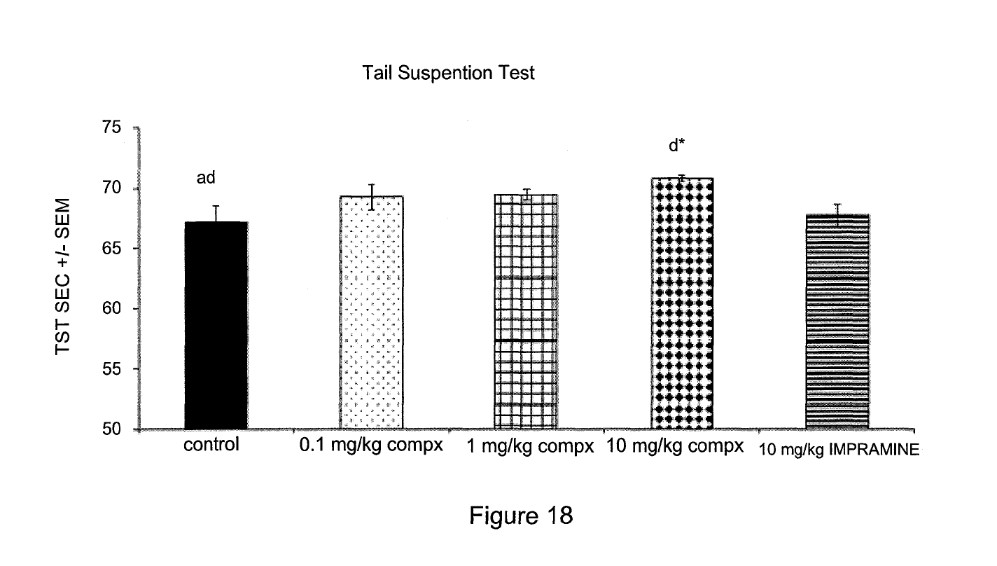

50, 2 month old BALB/c mice, 10 mice per group, on ad libitum diet were divided to 5 experimental groups with equal weight. On the 2, 3, 4, 5, 9, 11, 16, 18, 23, 25, 29, 31 days mice were administered with either vehicle or compound x 0.1, 1, 10 mg/kg i.p. or imipramine 10 mg/kg. Tail Suspension Test was evaluated on day 23 and FIG. 18 shows that 10 mg/kg improved significantly motor activity (p<0.05) more than impramine and compound X other concentrations.

Open Filed Test

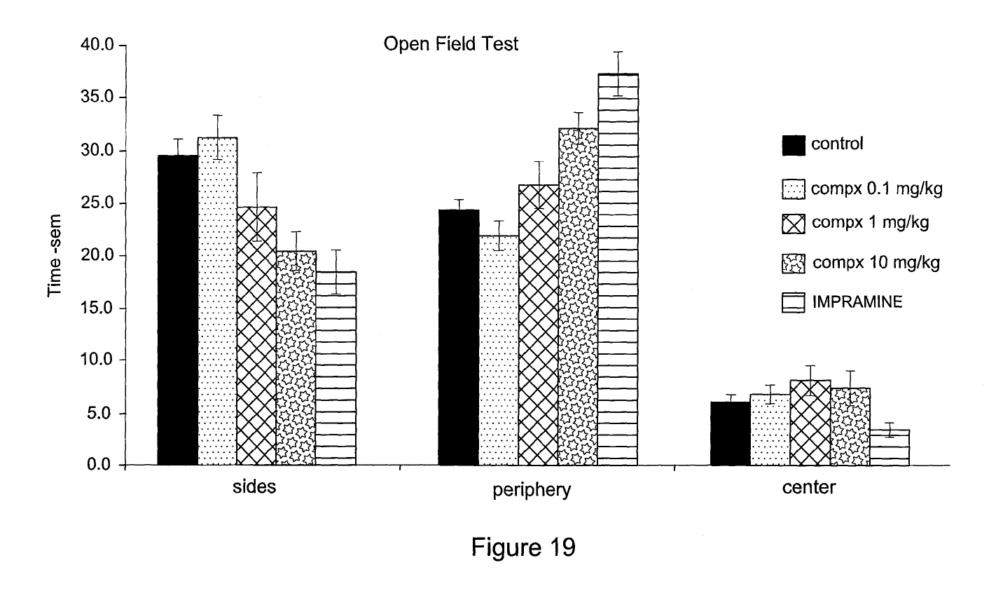

50, 2 month old BALB/c mice, 10 mice per group, on ad libitum diet were divided to 5 experimental groups with equal weight. On the 2, 3, 4, 5, 9, 11, 16, 18, 23, 25, 29, 31 days mice were administered with either vehicle or compound x 0.1, 1, 10 mg/kg i.p. or imipramine 10 mg/kg Open Field Test was evaluated on day 25. The more time they spent on the periphery the less depressed. FIG. 19 shows that 10 mg/kg of Compound X improved motor activity like the impramine and better than the other concentrations.

1-12. (canceled)

13. A method of treating depression comprising administering to a subject in need thereof a compound having the general formula (I): 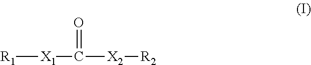
wherein
R₁ is selected from the group consisting of C₁₀-C₂₀ straight or branched alkyl, C₁₀-C₂₀ straight or branched alkenyl and C₁₀-C₂₀ straight or branched akynyl;
R₂ is C₂-C₈ straight or branched alkyl-OH; optionally substituted by at least one substituent selected from phenyl, phenyl-OH, —NH₂, —C(═O)O⁻, —NH₃⁺, —C(═O)NH₂, —SH, —SeH, —SCH₃, indolyl, toluyl, imidazoyl, pyrrolidinyl and guanidinyl;
X₁ and X₂ are each selected from the group consisting of a bond, —O—, —NH—; provided that at least one of X₁ and X₂ is —NH—.

14. A method according to claim 13, wherein R₁ is a straight C₁₀-C₂₀ alkenyl.

15. A method according to claim 13, wherein X₁ is a bond.

16. A method according to claim 13, wherein X₂ is a bond.

17. A method according to claim 13, wherein X₁ is —O— or —NH.

18. A method according to claim 13, wherein X₂ is —NH.

19. A method according to claim 13, wherein R₂ is a branched C₅-alkyl-OH.

20. A method according to claim 13, wherein R₂ is valinolyl.

21. A method according to claim 13, compound according to any one of the preceding claims selected from the group consisting of:
1-Oleoyl-L-Valinol amide,
Linoleoyl-L-Leucinol amide,
Oleoyl-L-Leucinol amide,
Elaidoyl L-Valinol amide,
Elaidoyl-D-Valinol amide,
Linoleoyl-L-Valinol amide,
Oleoyl-D-Valinol,
Oleyloxycarbonyl-L-Valinol amide,
Oleyloxycarbonyl-D-Valinol amide,
Oleyl-amine-carbonyl D-valinol,
Oleyl-amine-carbonyl L-valinol,
Oleylamino-D-2-hydroxyvaline amide, and
Oleoyl-L-phenyl alaninol.

22. A method according to claim 13, wherein said compound is administered in a dose of between about 0.1 mg/Kg to about 10 mg/Kg.
